(19) United States
(12) Patent Application Publication
(10) Pub. No.: US 2026/0301948 A1
(43) Pub. Date: Oct. 1, 2026

(54) AI Driven Additional Review of Medical Images

(71) Applicant: DeepHealth, Inc., Cambridge, MA (US)

(72) Inventors: Bryan T. Haslam, Merrimack, NH (US); Alma Gregory Sorensen, Harvard, MA (US); Benjamin S. Reece, Scottsdale, AZ (US)

(21) Appl. No.: 19/476,583

(22) PCT Filed: May 3, 2024
(86) PCT No.: PCT/US2024/027809

§ 371 Date: Oct. 17, 2025

Related U.S. Application Data
(60) Provisional application No. 63/464,430, filed on May 5, 2023.

(51) Int. Cl. G16H 50/20; G16H 15/00; G16H 30/40

(52) U.S. Cl. CPC G16H 50/20; G16H 15/00; G16H 30/40

(57) ABSTRACT

The present disclosure provides methods, systems, and non-transitory computer readable media for evaluating patient tissue screening images using an Al model as a tool for assisting physicians in correctly identifying malignancies in screening images. By generating an Al score based on a malignancy likelihood from the screening images, various workflows may follow to either validate or contradict a physician's decision for recalling the patient. Where the Al score contradicts the recall decision, the screening images are transmitted to a second physician for review, whereby the original recall decision is affirmed or a consultation between the physicians is requested.

### CROSS REFERENCE TO RELATED APPLICATIONS

This application claims the benefit of U.S. Provisional Patent Application Ser. No. 63/464,430, filed on May 5, 2023, and entitled “AI DRIVEN ADDITIONAL REVIEW OF MEDICAL IMAGES,” which is herein incorporated by reference in its entirety.

### STATEMENT REGARDING FEDERALLY SPONSORED RESEARCH

Not applicable.

### BACKGROUND

Breast cancer is the second leading cause of cancer mortality amongst women in the United States. As breast cancer progresses, it becomes harder and harder to treat as evidenced by a five-year relative survival rate of 93% for women with stage II cancer compared to 22% for those with stage IV cancer. The survival rate for late-stage cancer is even worse for some racial subgroups. Early detection is critical to diagnosing breast cancer and other forms of cancer before the disease becomes more likely to cause significant morbidity or mortality, and screening mammography is the tool for early detection. In the United States, although different societies have different recommendations, mammography screening is currently recommended either on an annual basis for women starting at the age of 40 or 45 years old or every two years. Overall, there are over 39 million mammograms performed each year in the US. This enormous screening effort highlights the medical and societal consensus that early detection of this potentially deadly disease is beneficial.

### SUMMARY

The effectiveness of screening mammography depends on the ability of radiologists to provide quality interpretations by recalling patients with cancer (sensitivity) while limiting the number of patients recalled without cancer (specificity). Close to 50% of radiologists have unacceptable interpretation performance either in terms of sensitivity or specificity. For example, the Breast Cancer Surveillance Consortium evaluated 323 MQSA-qualified radiologists; separately, the National Mammography Database (Lee et al. 2021) evaluated 1223 MQSA-qualified radiologists. Both evaluations showed unacceptable cancer detection rates (low sensitivity) and/or unacceptable recall rates (low specificity) among more than 40% percent of practitioners. The impact of this is that patients receive highly variable care.

Cancerous lesions are rare and often indicated by small, subtle features (e.g., microcalcifications, lesions partially masked by dense tissue, lesions at the edge of the image). Further contributing to variability is the practical reality that there is limited expertise available to interpret mammograms effectively. Estimates that, at most, 30% of screening mammograms are interpreted by breast imaging experts continue to mirror national practice. And even when experts are available, such experts can miss cancers. In many locations, especially rural locations, interpretation quality may be even worse given that screening mammograms are interpreted by general radiologists who only spend a limited amount of time interpreting mammography despite being MQSA-qualified. This problem of limited expertise is growing as the number of experts is not keeping pace with the need and the general shortage of radiologists is growing. Since experts can take up to a decade to train, it is not possible to solve this challenge any time soon through increased training of radiologists. The result is more and more mammograms are read by radiologists with unacceptable interpretation performance. The goal should be to provide expert-level interpretation equitably to all patients.

The differences between an expert-level interpretation, an acceptable interpretation, and an unacceptable interpretation are assessed by measuring cancer detection rate (CDR) and recall rate (RR). These measures correlate with the sensitivity and specificity of readers and thus correspond with the likelihood of whether cancers of individual patients will be missed or patients will undergo work-ups for non-cancers. The drawbacks of a missed cancer diagnosis are delayed diagnosis leading to increasing morbidity and mortality, while working up a patient without cancer causes patient anxiety, potential side effects of additional procedures and additional costs. Though the latter “harms” are less severe per patient, they are much more likely.

If 40% of interpreting physicians have unacceptable performance, as published estimates suggest (and some observers believe this estimate is overly forgiving of radiologists), then out of 30 million screening mammograms per year, well over 1 million unnecessary recalls are occurring every year, and more than 25,000 cancers are going undetected, due to poor quality interpretation.

Similar issues are associated with review and interpretation of images for screening other forms of cancer as well, for example, for lung cancer and prostate cancer. Additionally, in the pathology field, similar issues are associated with review and interpretation of images for patients in the form of digitally imaged or scanned pathology slides of patient tissue, including for breast tissue, lung tissue, prostate tissue, and other tissue areas.

The present disclosure addresses the aforementioned drawbacks by providing methods, systems and nontransitory computer readable media for evaluating breast tissue imagery using an artificial intelligence (AI) model.

In accordance with one aspect of the present disclosure, a method for evaluating patient tissue imagery using an AI model is described. The method includes receiving, using a processor, one or more images of tissue of a patient and transmitting the one or more images of the tissue to the AI model. The AI model generates an AI score output, wherein the AI score assesses the one or more images of tissue for the likelihood of one or more malignancies. The method further compares the AI score to a threshold to determine an AI recall score. The method further includes receiving, from a first radiologist via a first workstation, a radiologist recall score for the one or more images indicating an assessment by the first radiologist. The method further includes determining, based on the AI recall score and the radiologist recall score, whether to output the one or more images to a second workstation for review by a second radiologist, and generating an indication of the determination.

In accordance with another aspect of the present disclosure, a system for evaluating patient tissue imagery using an artificial intelligence (AI) model is described. The system includes a memory, a communication interface, and a processor coupled to the memory and to the communication interface. The processor is configured to receive one or more images of tissue of a patient and transmit the one or more images of the tissue to the AI model. The processor further generates an AI score output from the AI model, wherein the AI score assesses the one or more images of tissue for the likelihood of one or more malignancies and compares the AI score to a threshold to determine an AI recall score. The processor further receives via the communication interface, from a first radiologist via a first workstation, a radiologist recall score for the one or more images indicating an assessment by the first radiologist, and determines, based on the AI recall score and the radiologist recall score, whether to output the one or more images to a second workstation for review by a second radiologist. The processor further generates an indication of the determination.

In accordance with another aspect of the present disclosure, a nontransitory computer readable medium is described, comprising instructions that, when executed by a processor, cause the processor to receive one or more images of tissue of a patient and transmit the one or more images of the tissue to an artificial intelligence (AI) model. The instructions further cause the processor to generate an AI score output from the AI model, wherein the AI score assesses the one or more images of tissue for the likelihood of one or more malignancies and compare the AI score to a threshold to determine an AI recall score. The instructions further cause the processor to receive, from a first radiologist via a first workstation, a radiologist recall score for the one or more images indicating an assessment by the first radiologist. The instructions further cause the processor to determine, based on the AI recall score and the radiologist recall score, whether to output the one or more images to a second workstation for review by a second radiologist, and generate an indication of the determination.

### BRIEF DESCRIPTION OF THE DRAWINGS

FIG. 1 is a block diagram of an example system for evaluating breast tissue imagery using an AI model.

FIG. 2 is a block diagram of example components that can implement the system of FIG. 1.

FIG. 3 is a block diagram that shows an example x-ray imaging system.

FIG. 4 is a block diagram that shows an example embodiment of a model for generating regions of interest for a two-dimensional slice of three-dimensional tomosynthesis data.

FIG. 5 is a further block diagram that shows an example secondary model for generating an AI score based on a malignancy likelihood score.

FIG. 6 is a diagram of an example workstation used by a first and/or second radiologist.

FIG. 7 is a block diagram of an example processor for implementing the instructions of the breast malignancy imagery evaluation method, according to aspects of the present disclosure.

FIG. 8A is an example flowchart of the method according to aspects of the present disclosure.

FIG. 8B is an example logic flowchart of an aspect of the flowchart illustrated in FIG. 8A.

FIG. 8C is an example logic flowchart of an aspect of the flowchart illustrated in FIG. 8A.

FIG. 8D is an example logic flowchart of an aspect of the flowchart illustrated in FIG. 8A.

FIG. 8E is another example logic flowchart according to aspects of the present disclosure.

FIGS. 9A, 9B, 9C, 9D, and 10 illustrate experimental results according to some aspects of the disclosure.

### DETAILED DESCRIPTION

The present disclosure provides methods, systems and nontransitory computer readable media describing a workflow which allows evaluation of cancer screening images, such as, for example mammograms, with a higher cancer detection rate and lower recall rate. A higher cancer detection rate results in increased sensitivity and fewer false negative results. A lower recall rate results in increased specificity and fewer false positive results.

Described herein is the implementation of an AI model for assessing each patient tissue image data (e.g., breast tissue image data, lung tissue image data, prostate tissue image data, etc.) for suspicion of malignancies and assigning the exam for one or a plurality of workflows based on the suspicion of a malignancy as will be described in further detail below.

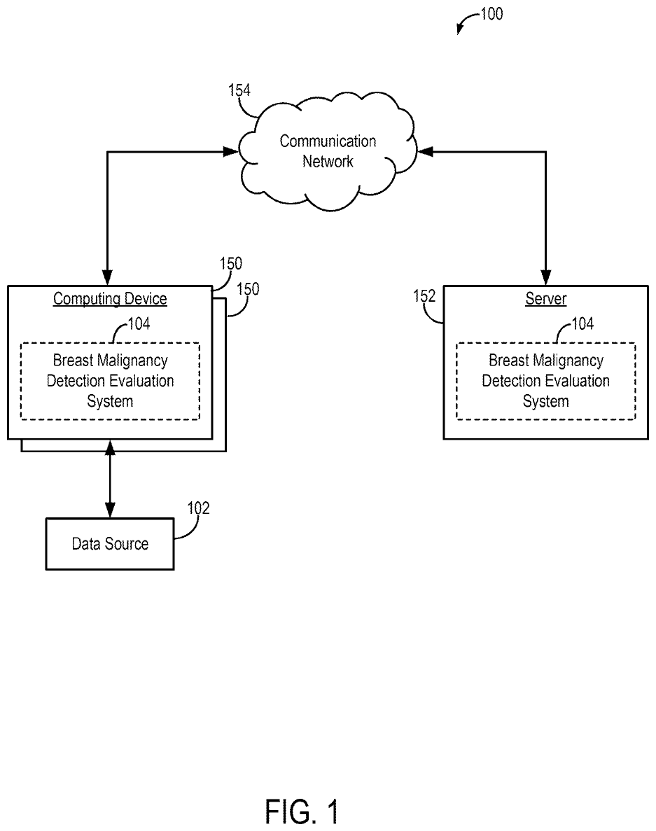

Referring now to FIG. 1, an example of a system 100 for evaluating cancer detection using an artificial intelligence (AI) algorithm in accordance with some embodiments of the systems and methods described in the present disclosure is shown. In some embodiments, the cancer data may include medical imaging data acquired from a subject's tissue (e.g., breast tissue, lung tissue, prostate tissue, etc.). As shown in FIG. 1, one or more computing devices 150 can receive one or more types of cancer imaging data (e.g., fluoroscopic imaging data, x-ray imaging data, computerized tomography (CT) imaging data, magnetic resonance imaging (MRI) data, ultrasound imaging data) from data source 102. In some embodiments, computing device 150 can execute at least a portion of a malignancy detection evaluation system 104 to classify patient tissue imaging data received from the data source 102 and/or to generate feature data or maps based on the tissue measurement data received from the data source 102.

Additionally or alternatively, in some embodiments, the computing device 150 can communicate information about data received from the data source 102 to a server 152 over a communication network 154, which can execute at least a portion of the malignancy detection evaluation system 104. In such embodiments, the server 152 can return information to the computing device 150 (and/or any other suitable computing device) indicative of an output of the malignancy detection evaluation system 104.

In some embodiments, computing device 150 and/or server 152 can be any suitable computing device or combination of devices, such as one or more desktop computers, laptop computers, smartphones, tablet computers, wearable computers, server computers, virtual machines being executed by a physical computing device, and so on. In an example, the computing device 150 may be the first workstation and second workstation, or a first computing device 150 may be the first workstation and a second computing device 150 may be the second workstation.

In some embodiments, the data source 102 can be any suitable source of data (e.g., measurement data, x-ray imaging data, computerized tomography (CT) imaging data, fluoroscopic imaging data, MRI data, ultrasound imaging data, images or maps reconstructed from such data), such as an X-ray system or other suitable imaging or functional measurement device, another computing device (e.g., a server storing data), and so on. In some embodiments, data source 102 can be local to computing device 150. For example, data source 102 can be incorporated with computing device 150 (e.g., computing device 150 can be configured as part of a device for capturing, scanning, and/or storing data). As another example, data source 102 can be connected to computing device 150 by a cable, a direct wireless link, and so on. Additionally or alternatively, in some embodiments, data source 102 can be located locally and/or remotely from computing device 150, and can communicate data to computing device 150 (and/or server 152) via a communication network (e.g., communication network 154).

In some embodiments, communication network 154 can be any suitable communication network or combination of communication networks. For example, communication network 154 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, and so on. In some embodiments, communication network 154 can be a local area network, a wide area network, a public network (e.g., the Internet), a private or semiprivate network (e.g., a corporate or university intranet), any other suitable type of network, or any suitable combination of networks. Communications links shown in FIG. 1 can each be any suitable communications link or combination of communications links, such as wired links, fiber optic links, Wi-Fi links, Bluetooth links, cellular links, and so on.

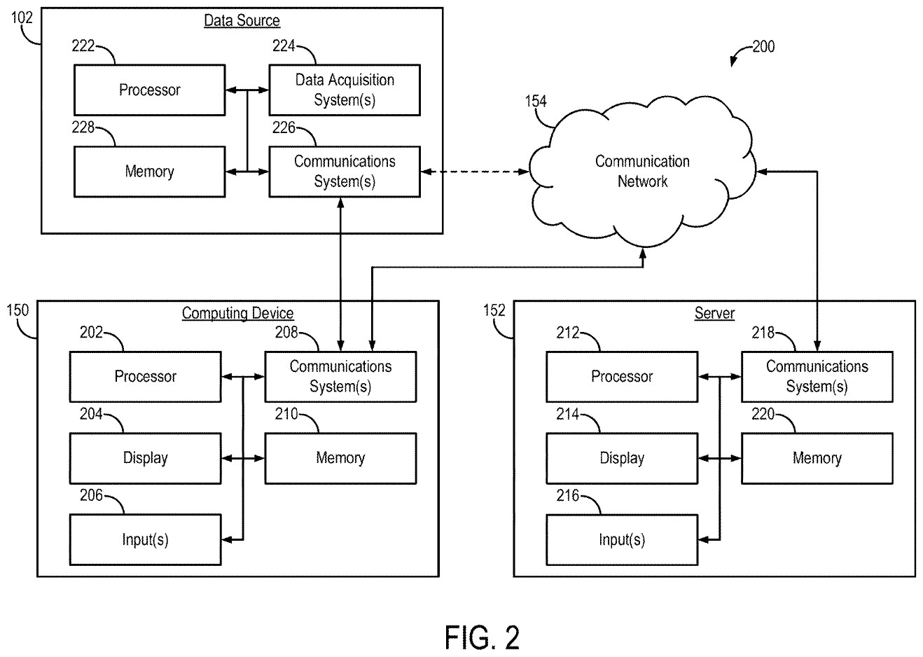

Referring now to FIG. 2, an example of hardware 200 that can be used to implement the data source 102, computing device 150, and server 152 in accordance with some embodiments of the systems and methods described in the present disclosure is shown. As shown in FIG. 2, in some embodiments, one or more computing devices 150 can include a processor 202, a display 204, one or more inputs 206, one or more communication systems 208, and/or memory 210. In some embodiments, processor 202 can be any suitable hardware processor or combination of processors, such as a central processing unit (“CPU”), a graphics processing unit (“GPU”), and so on. In some embodiments, display 204 can include any suitable display devices, such as a computer monitor, a touchscreen, a television, and so on. In some embodiments, inputs 206 can include any suitable input devices and/or sensors that can be used to receive user input, such as a keyboard, a mouse, a touchscreen, a microphone, and so on.

In some embodiments, communications systems 208 can include any suitable hardware, firmware, and/or software for communicating information over communication network 154 and/or any other suitable communication networks. For example, communications systems 208 can include one or more transceivers, one or more communication chips and/or chip sets, and so on. In a more particular example, communications systems 208 can include hardware, firmware and/or software that can be used to establish a Wi-Fi connection, a Bluetooth connection, a cellular connection, an Ethernet connection, and so on.

In some embodiments, memory 210 can include any suitable storage device or devices that can be used to store instructions, values, data, or the like, that can be used, for example, by processor 202 to present content using display 204, to communicate with server 152 via communications system(s) 208, and so on. Memory 210 can include any suitable volatile memory, non-volatile memory, storage, or any suitable combination thereof. For example, memory 210 can include RAM, ROM, EEPROM, one or more flash drives, one or more hard disks, one or more solid state drives, one or more optical drives, and so on. In some embodiments, memory 210 can have encoded thereon, or otherwise stored therein, a computer program for controlling operation of computing device 150. In such embodiments, processor 202 can execute at least a portion of the computer program to present content (e.g., images, heat maps, user interfaces, graphics, tables), receive content from server 152, transmit information to server 152, and so on.

In some embodiments, server 152 can include a processor 212, a display 214, one or more inputs 216, one or more communications systems 218, and/or memory 220. In some embodiments, processor 212 can be any suitable hardware processor or combination of processors, such as a CPU, a GPU, and so on. In some embodiments, display 214 can include any suitable display devices, such as a computer monitor, a touchscreen, a television, and so on. In some embodiments, inputs 216 can include any suitable input devices and/or sensors that can be used to receive user input, such as a keyboard, a mouse, a touchscreen, a microphone, and so on.

In some embodiments, communications systems 218 can include any suitable hardware, firmware, and/or software for communicating information over communication network 154 and/or any other suitable communication networks. For example, communications systems 218 can include one or more transceivers, one or more communication chips and/or chip sets, and so on. In a more particular example, communications systems 218 can include hardware, firmware and/or software that can be used to establish a Wi-Fi connection, a Bluetooth connection, a cellular connection, an Ethernet connection, and so on.

In some embodiments, memory 220 can include any suitable storage device or devices that can be used to store instructions, values, data, or the like, that can be used, for example, by processor 212 to present content using display 214, to communicate with one or more computing devices 150, and so on. Memory 220 can include any suitable volatile memory, non-volatile memory, storage, or any suitable combination thereof. For example, memory 220 can include RAM, ROM, EEPROM, one or more flash drives, one or more hard disks, one or more solid state drives, one or more optical drives, and so on. In some embodiments, memory 220 can have encoded thereon a server program for controlling operation of server 152. In such embodiments, processor 212 can execute at least a portion of the server program to transmit information and/or content (e.g., data, images, a user interface) to one or more computing devices 150, receive information and/or content from one or more computing devices 150, receive instructions from one or more devices (e.g., a personal computer, a laptop computer, a tablet computer, a smartphone), and so on.

In some embodiments, data source 102 can include a processor 222, one or more data acquisition system(s) or inputs 224, one or more communications systems 226, and/or memory 228. In some embodiments, processor 222 can be any suitable hardware processor or combination of processors, such as a CPU, a GPU, and so on. In some embodiments, the one or more inputs 224 are generally configured to acquire data and can include a functional lumen imaging probe. Additionally or alternatively, in some embodiments, one or more inputs 224 can include any suitable hardware, firmware, and/or software for coupling to and/or controlling operations of a functional lumen imaging probe. In some embodiments, one or more portions of the one or more inputs 224 can be removable and/or replaceable.

The data source 102 can also include additional inputs and/or outputs. For example, data source 102 can include input devices and/or sensors that can be used to receive user input, such as a keyboard, a mouse, a touchscreen, a microphone, a trackpad, a trackball, and so on. As another example, data source 102 can include any suitable display devices, such as a computer monitor, a touchscreen, a television, etc., one or more speakers, and so on.

In some embodiments, communications systems 226 can include any suitable hardware, firmware, and/or software for communicating information to computing device 150 (and, in some embodiments, over communication network 154 and/or any other suitable communication networks). For example, communications systems 226 can include one or more transceivers, one or more communication chips and/or chip sets, and so on. In a more particular example, communications systems 226 can include hardware, firmware and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 228 can include any suitable storage device or devices that can be used to store instructions, values, data, or the like, that can be used, for example, by processor 222 to control the one or more inputs 224; to receive data from the one or more inputs 224; to generate images, heat maps, and/or computed parameters from data; to present content (e.g., images, heat maps, a user interface) using a display; to communicate with one or more computing devices 150; and so on. Memory 228 can include any suitable volatile memory, non-volatile memory, storage, or any suitable combination thereof. For example, memory 228 can include RAM, ROM, EEPROM, one or more flash drives, one or more hard disks, one or more solid state drives, one or more optical drives, and so on. In some embodiments, memory 228 can have encoded thereon, or otherwise stored therein, a program for controlling operation of data source 102. In such embodiments, processor 222 can execute at least a portion of the program to compute parameters, transmit information and/or content (e.g., data, images, heat maps) to one or more computing devices 150, receive information and/or content from one or more computing devices 150, receive instructions from one or more devices (e.g., a personal computer, a laptop computer, a tablet computer, a smartphone, etc.), and so on.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

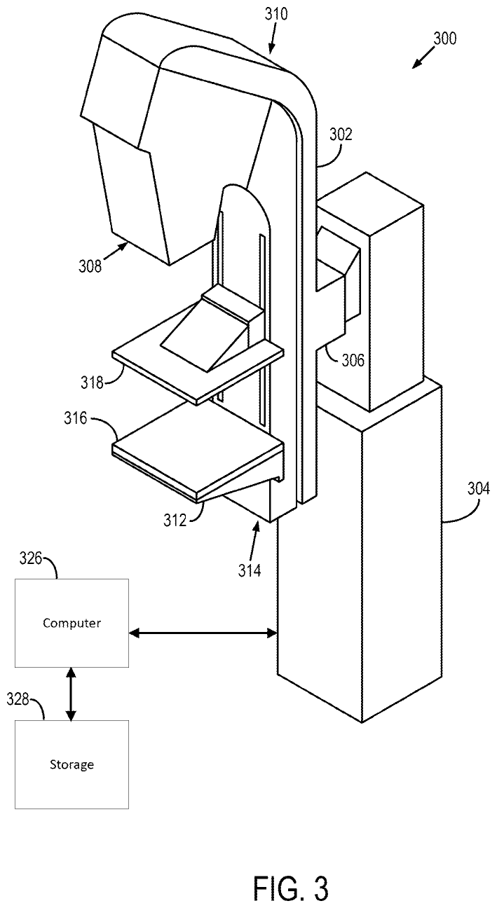

An example data source 102 is shown in FIG. 3. An x-ray imaging system 300, such as, for example, a three-dimensional (3D) digital breast tomosynthesis (DBT), can include an x-ray source assembly 308 coupled to a first end 310 of an arm 302. An x-ray detector assembly 312 can be coupled proximate an opposing end 314. The x-ray source assembly 308 may extend substantially perpendicular to the arm 302 and be directed toward the x-ray detector assembly 312. The x-ray detector assembly 312 also extends from the arm 302 such that the x-ray detector assembly 312 receives x-ray radiation produced by the x-ray source assembly 308, transmitted through the breast, and incident on the x-ray detector assembly 312. A breast support plate 316, and a breast compression plate 318, are positioned between the x-ray source assembly 308 and the x-ray detector assembly 312. The x-ray source assembly 308 may be stationary or movable. The x-ray imaging system 300 can generate a reconstructed image including 3D DBT data. The 3D DBT data can include a number of two-dimensional (2D) slices. In some embodiments, the reconstructed image can include 3D tomography data including a plurality of 2D slices having a thickness of about 1 mm. The 2D slices can be used to create a synthetic 2D image, which will be described below. In some embodiments, the x-ray imaging system 300 can generate intermediate two-dimensional “slabs,” representing, for example, maximum intensity projections of a subset of 2D slices. For example, a single maximum intensity projection slab can be generated from ten slices, and multiple slabs can be generated from a plurality of slices, such as ten slabs from one hundred slices. The reconstructed image can be applied as an input to a computer 326 which stores the image in a mass storage device 328, which can include a memory. The computer 326 may also provide commands to the x-ray imaging system 300 in order to control the generation of the reconstructed image. In some examples, the x-ray imaging system 300 is configured to capture x-ray images of other areas of a patient, for example, a patients lung(s) or prostate.

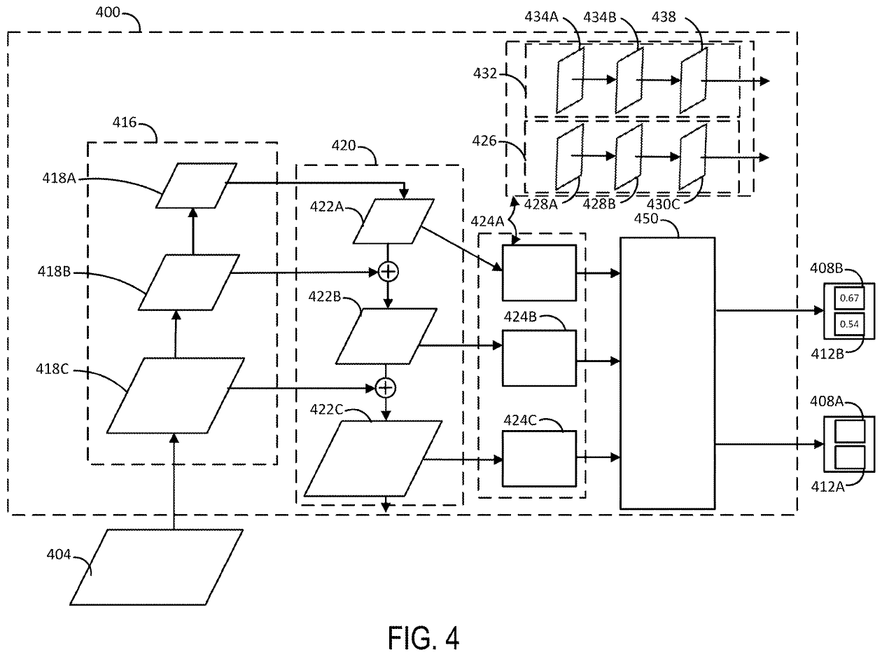

Referring to FIG. 4, an example embodiment of a model 400 for generating regions of interest (ROIs) for a two-dimensional (2D) slice 404 of three-dimensional (3D) tomosynthesis data is shown. The ROIs may be referred to as indicators. The model 400 can accept the 2D slice 404 and output any number of ROIs, for example, a first ROI including a first area 408A and a first score 408B, and a second ROI including a second area 412A and a second score 412B. Each ROI can be associated with a slice number indicating the 2D slice that the ROI was generated based on, for example a fourth slice of a set of seventy-five 2D slices. The slice number can be used when selecting and/or combining ROIs to create a synthetic image, as will be explained below. Depending on the characteristics of the 2D slice 404, such as when the 2D slice 404 is deemed to not have any regions that sufficiently indicate a potential malignancy, zero ROIs may be output. Each ROI can include an area that can be a subregion of the 2D slice 404. As noted above, each 2D slice can be formatted as an array of pixels. The subregion can be a subset of the array of pixels. In some embodiments, the model 400 can include one or more neural networks configured to detect objects within 2D images. The objects can be ROIs.

In some embodiments, the model 400 can output ROIs that follow a predetermined shape. For example, rectangular bounding boxes can be used to encompass a potential candidate for a tumor or lesion. It is contemplated that irregular shapes (e.g., a “blob” of pixels) can be used to better outline potential tumors or lesions. When creating a training database of ROIs, one or more human practitioners may find it more intuitive to use rectangular bounding boxes than other shapes. Neural networks that use segmentation mask-based approaches to identify objects could be used to output predicted ROIs with irregular shapes. The model 400 can then be trained to identify rectangular-shaped ROIs including a subarray of the pixels included in the 2D slice 404. The pixels of the ROI can include one or more color intensity values (e.g., a white intensity value) and a location within the 2D slice 404 (e.g., the pixel at a given (x, y) location in a 2000×1500 pixel slice). While some mammography imaging systems produce greyscale images of breast tissue, it is appreciated that the model can be used with colorized 2D slices. Each 2D slice of 3D tomosynthesis data can be the same size, such as 2000×1500 pixels.

In addition to the subarray of pixels, the ROI can include a relevancy score indicating how relevant the subarray of pixels is to determining a malignancy likelihood score. The relevancy score can be used to create a synthetic 2D image using one or more ROIs, as will be explained in detail below. The relevancy score can be selected from a range of values such as between 0 and 1. When identifying ROIs for a training dataset, a human practitioner can assign relevancy scores for each ROI within the range of the values. The human practitioner could alternatively assign relevancy scores using a different scale, such as 0-100 (with higher scores indication higher potential for malignancy) which could then be normalized to relevancy score range used by the model 400. In some embodiments, the human practitioner can identify ROIs as benign in order to better train the model 400 to identify potentially malignant ROIs.

In some embodiments, the model 400 can include a neural network such as a convolutional neural network. In order to train the model 400, a training dataset including 2D data consisting of full-field digital mammography (FFDM) images and/or slices from a set of 3D tomosynthesis images and pre-identified (e.g., by one or more medical practitioners) ROIs can be used to train the model. Human practitioners can identify ROIs by examining a given 2D image, outlining, using a predetermined shape such as a rectangular box, any regions that may be of interest, and assign a relevancy score to the predetermined shape based on their medical expertise and/or experience in evaluating tumors and/or lesions. Alternatively, the relevancy score can be assigned based on pathology results that indicate whether or not a lesion is malignant. A large training database can be generated by having one or more medical practitioners identify (e.g., annotate) ROIs in 2D images taken from a plurality of FFDM images or slices of 3D tomosynthesis images (e.g., images of multiple patients). An advantage of using FFDM images is that there are presently more publicly available annotated FFDM images than annotated 3D tomosynthesis images. Additionally, 2D images are easier to annotate than 3D tomosynthesis images, which can require annotating a large number of individual slices included in each 3D tomosynthesis image. Once trained, the model 400 can receive an input 2D slice and output one or more ROIs, each ROI including an estimated relevancy score and a subarray of pixels of the input 2D slice.

The model 400 can include a number of layers such as convolutional layers. It is understood that some embodiments of the model 400 may have different numbers of layers, a different arrangement of layers or other differences. However, in all embodiments, the model 400 can be capable of receiving an input 2D input slice and outputting any regions of interest associated with the input 2D input slice. The model 400 can be a one-stage detection network including one or more subnetworks.

The model 400 can include a first subnetwork 416. The first subnetwork 416 can be a feedforward residual neural network (“ResNet”) with one or more layers 418A-C. A second subnetwork 420 can be built on top of the first subnetwork to effectively create a single neural network, using the first subnetwork 416 as the backbone for the network. The second subnetwork 420 can contain a plurality of layers including a first layer 422A, a second layer 422B, and a third layer 422C, though other numbers of layers (e.g., five layers) can be used, and three layers are shown for simplicity. Each of the first layer 422A, the second layer 422B, and the third layer 422C can be a convolutional layer. Each layer can be made of a number of building blocks (not shown). Each building block can include a number of parameters layers such as three parameter layers, each parameter layer including a number of filters (e.g., 456) with a given filter size (e.g., 3×3). Each of the first layer 422A, the second layer 422B, and the third layer 422C can have an associated output size such as 144×144, 72×72, and 36×36. The output sizes can vary between input slices based on pre-processing conditions and/or parameters. As the output size decreases between layers of the second subnetwork 420, the number of filters of the parameter layers can increase proportionally, i.e., halving output size results in doubling the number of filters. The second subnetwork can also include a global average pooling layer connected to a final layer (i.e., the third layer 422C), a fully-connected layer connected to the global average pooling layer, and a softmax layer connected to the fully-connected layer and having a 1×1 output size (i.e., a single value).

The model 400 can include a plurality of tertiary subnetworks such as a first tertiary network 424A, a second tertiary network 424B, and a third tertiary network 424C. Each of the tertiary networks 424A-C can be connected to a layer of the second subnetwork 420. The first tertiary network 424A can be connected to the first layer 422A, the second tertiary network 424B can be connected to the second layer 422B, and the third tertiary network 424C can be connected to the third layer 422C. Each tertiary network can receive features from a layer of the second subnetwork 420 in order to detect tumors and/or lesions at different levels of scale.

Each tertiary network can include a box regression subnetwork 426. The box regression subnetwork 426 can include one or more convolutional layers 428A-B, each followed by rectified linear (ReLU) activations, and a final convolutional layer 430 configured to output regression coordinates corresponding to anchors associated with a portion of one of the layers of the second subnetwork 420 (and corresponding to an array of pixels of the input 2D slice 404). The anchors can be predetermined subarrays of the various layers of the second subnetwork 420. The regression coordinates can represent a predicted offset between an anchor and a predicted bounding box. For each bounding box included in an ROI, a set of regression coordinates (e.g., four regression coordinates) and the corresponding anchor can be used to calculate the coordinates of the bounding box.

Each tertiary network can include a classification subnetwork 432. The classification subnetwork 432 can include one or more convolutional layers 434A-B, each followed by ReLU activations, and a final convolutional layer 438 followed by sigmoidal activations to output predictions of object presence (i.e., malignant tumor and/or lesion presence). The classification subnetwork 432 can be used to obtain one or more estimations of whether or not a patient has a malignant tumor and/or lesion at various spatial locations of the 2D slice 404. More specifically, each bounding box can be associated with an estimated score output by the classification subnetwork. In some embodiments, the value of each estimated score can range from zero to one. One of the spatial locations can include an entire layer, i.e., first layer 422A, of the second subnetwork 420. In this way, the classification subnetwork 432 can output an estimation of whether or not a patient has a malignant tumor and/or lesion based on a 2D slice. It is contemplated that the final convolutional layer 438 can be followed by Softmax activations in models that are trained to classify multiple types of malignant regions, for example multiple levels of malignancy (e.g., low risk regions, high risk regions, etc.).

The model 400 can include an output layer 450 for normalizing data across different scales, calculating bounding box coordinates, and filtering out low scoring bounding box predictions. The output layer 450 can receive outputs from the tertiary subnetworks 424A-C and output one or more ROIs, each ROI including an array of pixels scaled to the array size of the 2D slice 404 and an associated score. The array of pixels can be a bounding box (e.g., a rectangular bounding box) calculated based on the regression coordinates and the anchors. The output layer 450 can filter out any scores below a predetermined threshold, for example, 0.5. In some embodiments, the output layer 250 can receive outputs from the tertiary subnetworks 424A-C and output a single malignancy likelihood score. In some embodiments, the single malignancy likelihood score can be selected to be the highest scoring bounding box score.

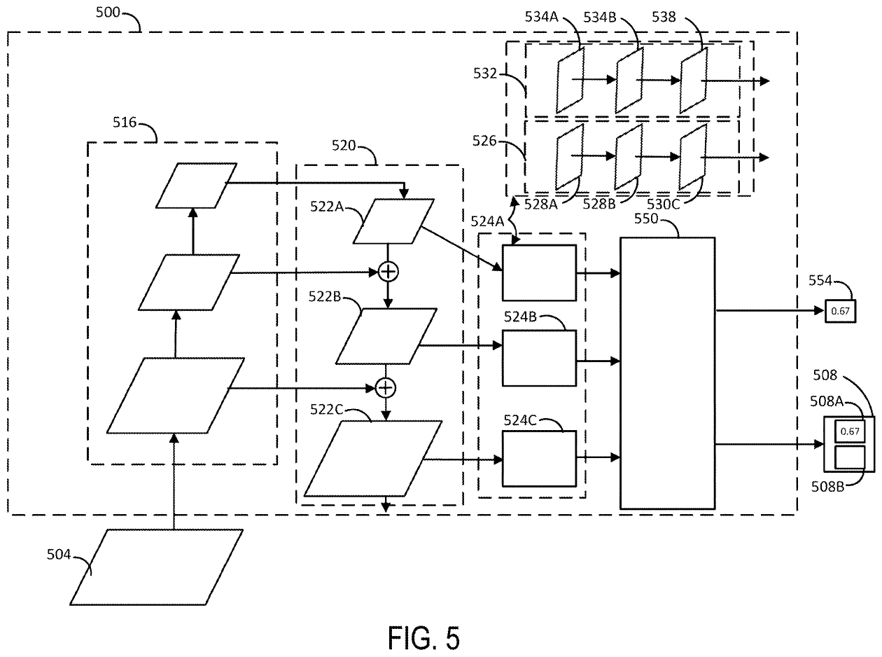

Referring to FIG. 5, an example secondary model 500 for generating a malignancy likelihood score 554 is shown. In some embodiments, the malignancy likelihood score 554 can indicate a category of risk, i.e., a low risk, medium risk, or high risk category. In some embodiments, the malignancy likelihood score 554 can be selected from a range of values, such as the integers 1-5, with 1 indicating a lowest risk level and 5 indicating a highest risk level.

In some embodiments, the secondary model 500 can include a neural network such as, for example, a residual convolutional neural network. In order to train the secondary model 500, a training dataset including synthetic images labeled as malignant or non-malignant can be used to train the model. Human practitioners can label the synthetic images. For instance, a synthetic image corresponding to a patient known to have cancer could be given a label of “1”, whereas a synthetic image corresponding to a patient known to not have cancer could be given a label of “0”. Once trained, the secondary model 500 can receive an input synthetic image and output a malignancy likelihood score indicating whether or not the breast tissue contains malignant tumors and/or lesions.

The secondary model 500 can include a number of layers such as convolutional layers. It is understood that some embodiments of the secondary model 500 may have different numbers of layers, a different arrangement of layers or other differences. However, in all embodiments, the secondary model 500 can be capable of receiving an input 2D synthetic image and outputting a malignancy likelihood score. The secondary model 500 can be a one-stage detection network including one or more subnetworks.

Briefly referring back to FIG. 4 as well as FIG. 5, the secondary model 500 can include a primary subnetwork 516 and a secondary subnetwork 520, which can be the same as the first subnetwork 416 and the second subnetwork 420 of the model 400 described above. In some embodiments, the primary subnetwork 516 and the secondary subnetwork 520 can be the same as the first subnetwork 416 and the second subnetwork 420 after the model 400 has been trained. In other words, the secondary model 500 can be initialized with weights from the model 400 before training.

The secondary model 500 is important because the model 400 described above may be able to detect regions of the breast tissue that are of interest but may not be able to accurately determine if the ROIs are actually malignant. The secondary model 500 may be used to more accurately estimate malignancy of the breast tissue using the synthetic images generated by the model 400 described above.

The model 500 can include a plurality of tertiary subnetworks, such as a first tertiary network 524A, a second tertiary network 524B, and a third tertiary network 524C. Each of the tertiary networks 524A-C can be connected to a layer of the secondary subnetwork 520. The first tertiary network 524A can be connected to a first layer 522A, the second tertiary network 524B can be connected to a second layer 522B, and the third tertiary network 524C can be connected to a third layer 522C. Each tertiary network can receive features from a layer of the secondary subnetwork 520 in order to estimate malignancy of the breast tissue at different levels of scale.

Each tertiary network can include a box regression subnetwork 526. The box regression subnetwork 526 can include one or more convolutional layers 528A-B, each followed by rectified linear (ReLU) activations, and a final convolutional layer 530 configured to output regression coordinates corresponding to anchors associated with a portion of one of the layers of the secondary subnetwork 520 (and corresponding to an array of pixels of the input synthetic 2D slice 504). The anchors can be predetermined subarrays of the various layers of the secondary subnetwork 520. The regression coordinates can represent a predicted offset between an anchor and a predicted bounding box. For each bounding box included in an ROI, a set of regression coordinates (e.g., four regression coordinates) and the corresponding anchor can be used to calculate the coordinates of the bounding box.

Each tertiary network can include a classification subnetwork 532. The classification subnetwork 532 can include one or more convolutional layers 534A-B, each followed by ReLU activations, and a final convolutional layer 538 followed by sigmoidal activations to output predictions of object presence (i.e., malignant tumor and/or lesion presence). The classification subnetwork 532 can be used to obtain one or more estimations of whether or not a patient has a malignant tumor and/or lesion at various spatial locations of the synthetic 2D slice 504. More specifically, each bounding box can be associated with an estimated score output by the classification subnetwork 532. The bounding box can also be associated with a slice number as described above. In some embodiments, the value of each estimated score can range from zero to one. One of the spatial locations can include an entire layer, i.e., first layer 522A, of the secondary subnetwork 520. In this way, the classification subnetwork 532 can output an estimation of whether or not a patient has a malignant tumor and/or lesion based on a 2D slice. It is contemplated that the final convolutional layer 538 can be followed by Softmax activations in models that are trained to classify multiple types of malignant regions, for example multiple levels of malignancy (e.g., low risk regions, high risk regions, etc.).

The model 500 can include an output layer 550 for normalizing data across different scales, calculating bounding box coordinates, and filtering out low scoring bounding box predictions. The output layer 550 can receive outputs from the tertiary subnetworks 524A-C and output one or more ROIs, each ROI including an array of pixels scaled to the array size of the 2D slice 504 and an associated score. The array of pixels can be a bounding box (e.g., a rectangular bounding box) calculated based on the regression coordinates and the anchors. The output layer 550 can filter out any scores below a predetermined threshold, for example, 0.5. After filtering, the output layer 550 can determine the array of pixels of each ROI based on the one or more anchors associated with the remaining scores. The output layer 550 may resize the anchors in order to match the scale of the 2D slice 504, as may be necessary for anchors associated with smaller layers of the secondary subnetwork 520, before including the anchor as the array of an output ROI. The output layer 550 can receive outputs from the tertiary subnetworks 524A-C and output the malignancy likelihood score 554. In some examples, the malignancy likelihood score 554 may be numerical and defined by a range (e.g., from 0 to 10, from 0 to 100, etc.) in which a higher number indicates a malignancy is more likely. In some examples, the malignancy likelihood score 554 is an example of an AI score output by an AI model. In some embodiments, the malignancy likelihood score 554 can be selected to be the highest scoring bounding box score. In some embodiments, the model 500 can output one or more ROIs 508, each including a score 508A and an array of pixels 508B. The array of pixels 508B can be a rectangular bounding box. The one or more ROIs 508 can provide additional information to a practitioner about potentially malignant regions of the synthetic image 504.

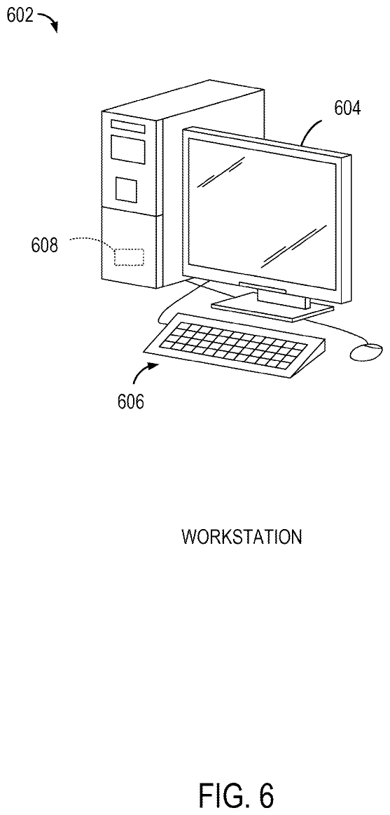

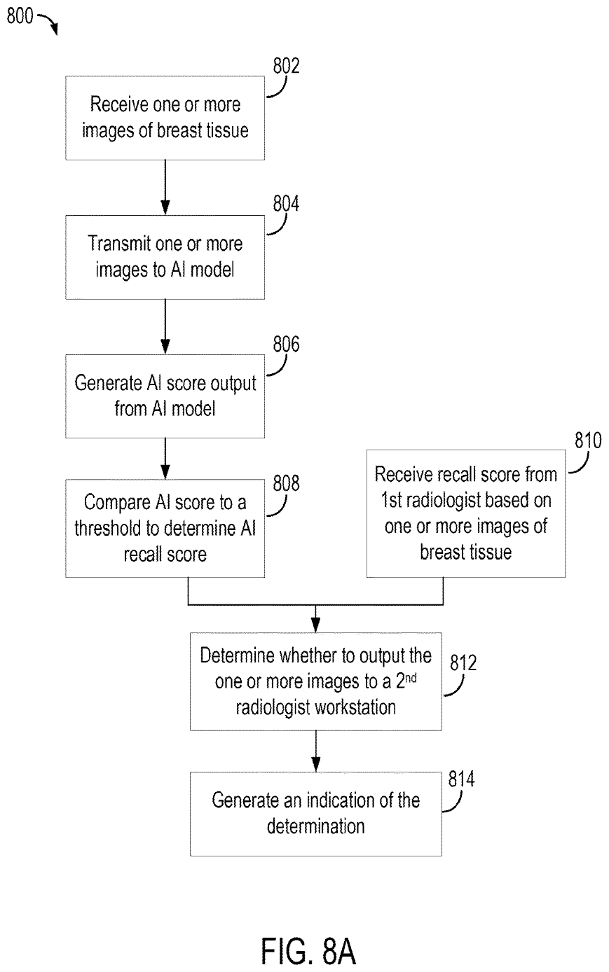

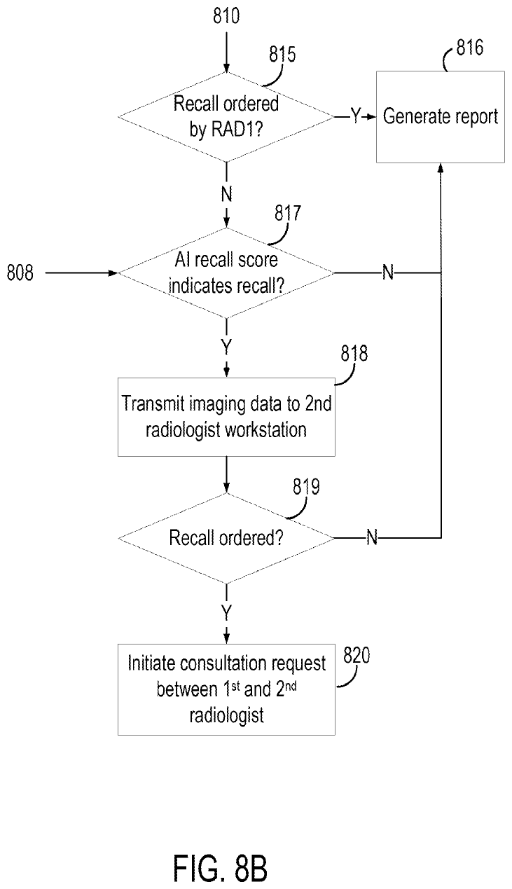

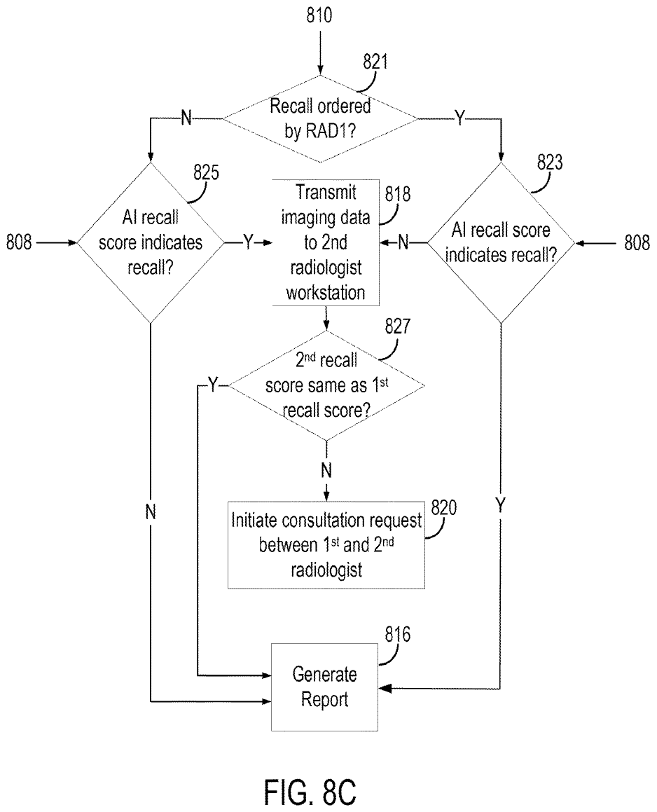

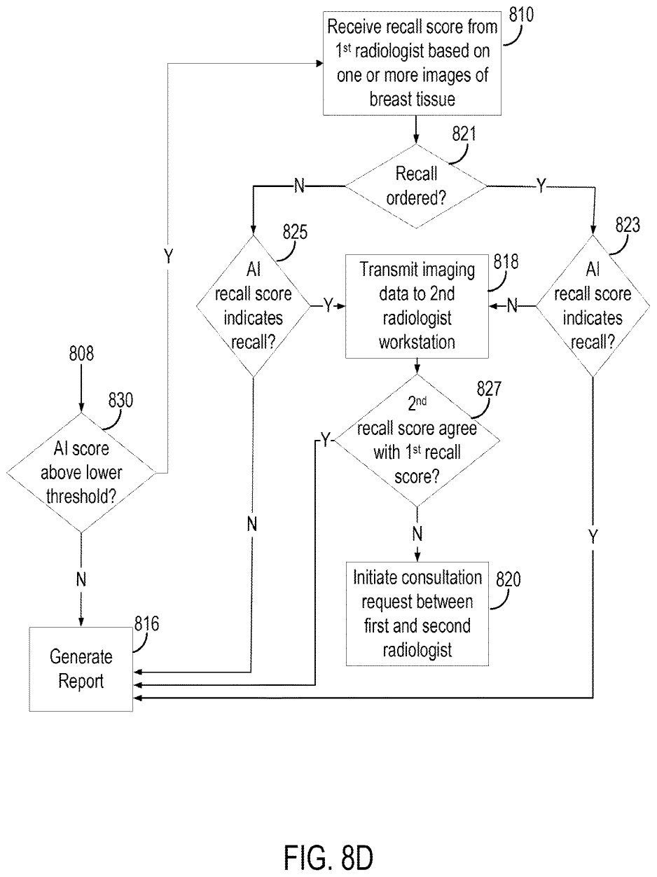

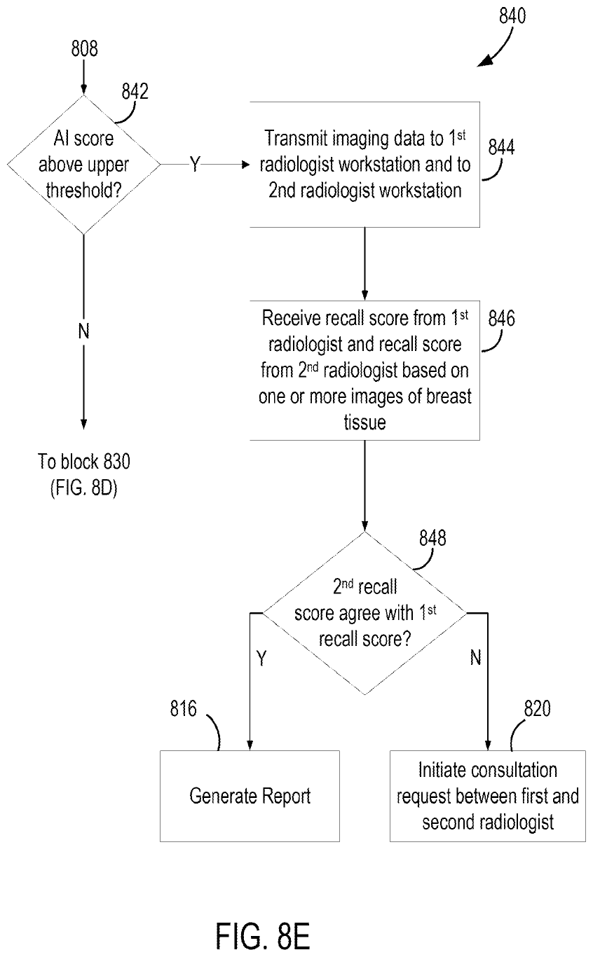

Referring now to FIG. 6, an example of a workstation 602 is shown. The workstation 602 is an example of a first and second workstation operated by a first and second radiologists, respectively (e.g., in the processes of FIGS. 8A-8D). In an example, the workstation 602 may include a display 604; one or more input devices 606, such as a keyboard and mouse; and a processor 608. The processor 608 may include a commercially available programmable machine running a commercially available operating system. In an example, the workstation 602 provides a first and/or second radiologists interface that can i) receive imaging data (e.g., from the data source 102), ii) receive a request to display imaging data, iii) display received imaging data, iv) receive and output requests for radiologist assessment of imaging data, and/or v) receive entry of a recall score from an assessing radiologist.

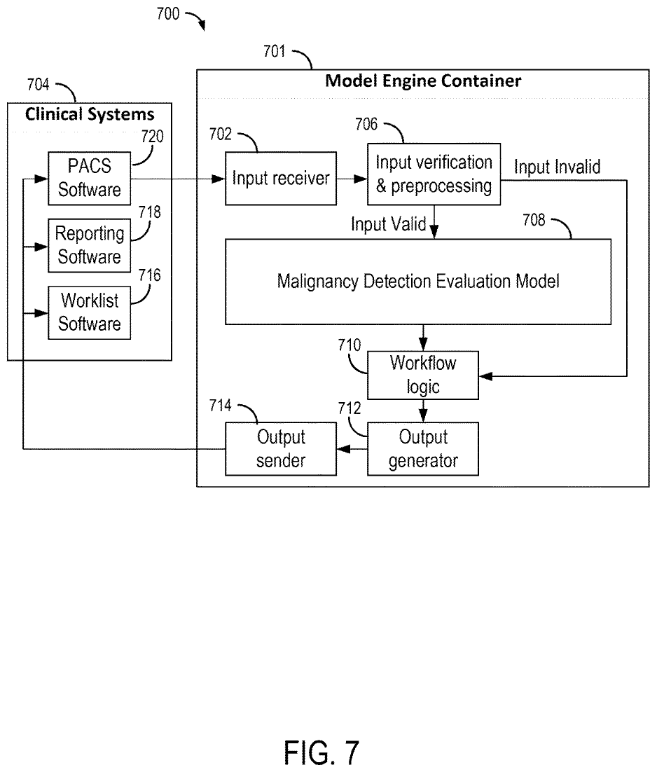

FIG. 7 shows an example of a software architecture design 700 of the malignancy detection evaluation system 104. The software architecture design 700 may include a model engine container 701 and clinical systems 704. An input receiver module 702 of the model engine container 701 may receive input data (e.g., mammogram images) from the clinical systems 704 (e.g., PACS software 720) and stores the input data in an internal storage location for subsequent processing by other modules. In some examples, the input receiver module 702 includes a component called a listener, which can continuously “listen” for new DICOM studies sent via the customer's clinical systems 704 (e.g., PACS software 720) and save the studies to a dedicated folder, pre-specified during installation, that is accessible by a docker container. The input receiver module 702 may place the received studies in a queue to be processed by an input verification & preprocessor module 706 of the model engine container 701.

The input verification & preprocessor module 706 can perform a series of acceptance criteria checks to determine whether or not each mammogram (or other images of patient tissue) is suitable for analysis. The module 706 can then extract the pixel data and relevant DICOM attributes. This step can ensure that the model evaluates mammography studies for which the system is indicated and configured. This step mitigates the risk of the software evaluating an exam that could give unexpected results or an exam that was not intended as a screening mammogram for clinical interpretation.

After verification and preprocessing, the input verification & preprocessor module 706 may provide a mammogram study (or other image(s) of patient tissue) to the malignancy detection evaluation model 708. In an example, the model 708 may include models 400 and 500 described above.

The workflow logic module 710 can combine the results from the model to determine to which workflow to assign the study, also referred to as the exam. Some examples of workflows are described in further detail below with respect to FIGS. 8A-D.

The output generator 712 creates user outputs. The user outputs can include the workflow assignment for each exam, a Mammo CAD SR file for each exam, and a Breast Imaging Repository, and/or Data System (BIRADS) output for exams assigned to the “No Further Review” workflow. If the input is invalid, the outputs may not be generated.

The outputs sender module 714 is configurable to route the outputs to one or more clinical systems 704. This configuration may occur at the time of installation to ensure the user outputs correctly interface with the clinical systems 704. In an example, the clinical systems 704 may include and execute worklist software 716, reporting software 718, and PACS software 720.

In an example, the model engine container, including components 702, 706-714, may be an example of the system 104 of FIG. 1. In some examples, one or more of the components 702, 706-714 include program instructions (e.g., software), including machine readable code stored on a nontransitory computer readable medium, executable by one or more electronic processors to implement the functionality of each component. In some examples, one or more of the components 702, 706-714 include one or more electronic processors (e.g., microcontrollers, microprocessors, field programmable gate arrays (FPGAs), application specific integrated circuits (ASICs) incorporating and/or configured to execute or implement the program instructions of the components 702, 706-714. For example, the program instructions of the one or more components 702, 706-714 may be stored on and executed by an electronic processor of the computing device(s) 150 and/or the server 162 of FIG. 1. Additionally, in an example, the clinical systems 704 may be implemented by or across one or more of the computer devices 150 and the server 152 of FIG. 1.

FIG. 8A shows an example of a method 800 for evaluating patient tissue imagery using an AI model, according to aspects of the present disclosure. The method 800 is described with respect to breast tissue imagery; however, the method 800 similarly applies to patient images of other types of patient tissue including lung tissue, prostate tissue, and other tissue types. Further, the method 800 similarly applies to patient images in the form of digitally imaged or scanned pathology slides of patient tissue, including breast tissue, lung tissue, prostate tissue, and other tissue types. In such examples, pathologists may be associated with the method 800 in place of the radiologists referenced. In some examples, the method 800 may be executed by the system 100 of FIG. 1, or more particularly by one or more electronic processors thereof (e.g., the processor 202 and/or the processor 212 illustrated in FIG. 2). In some examples, the method 800 may be executed by the system 100 implementing the design 700 for the malignancy detection evaluation system 104 as illustrated in FIG. 7. Additionally, although the blocks of the method 800 are illustrated in a particular order, in some examples, one or more of the blocks of the method 800 may be executed in parallel or partially in parallel with one another, in a different order than illustrated, or bypassed.

At block 802, the method includes receiving one or more images of breast tissue of a patient. The images may be received from a data source (e.g., the data source 102). Accordingly, the images may be received from an imaging device (see, e.g., FIG. 3) or from an intermediate storage device. The one or more images may be received by a processor (e.g., the processor 202 or 212) or a memory associated therewith (e.g., in communication with the processor).

At block 804, the one or more images are transmitted to the AI model for generating an AI score at block 806. For example, the processor 202 or 212 may transmit the one or more images to the AI model (e.g., the system 104 or model 708, which may, e.g., implement the models 400 and 500). For example, the processor 202 or 212 may transmit the one or more images by retrieving and transmitting the one or more images from a coupled memory (e.g., memory 210 or 220) to the AI model or by transmitting a request to the data source 102 to transmit the one or more images to the AI model. In response, the AI model generates or outputs the AI score. The output AI score may be received by the processor 202 or 212. In an example, the AI score is based on the likelihood of a malignancy in the images. In an example, the AI score is a continuous numerical value. For example, the AI score may be the score 554 output by the model 500 of FIG. 5. In some examples, the AI score is part of AI output that is output by the AI model based on processing the imaging data. For example, the AI output may include the aforementioned AI score (e.g., the AI score 554), and may also include localization information corresponding to the imaging data. The localization information may include, for example, one or more ROIs identified in the imaging data by the model 500.

At block 808, the AI score is compared to one or more thresholds to determine an AI recall score. In an example, the one or more thresholds are numerical values or ranges. In some examples, the processor (e.g., the processor 202 or 212) retrieves the one or more thresholds from a memory (e.g., the memory 210 or 220) and performs the comparison(s). In an example, the AI recall score is a binary metric indicating whether the AI model indicates a suggestion for recalling the patient for additional imaging (because of a high likelihood of malignancy) or a suggestion for not recalling the patient (because of a low likelihood of malignancy). Accordingly, in some examples, when the processor determines that the AI score is above the threshold, the processor may determine that the AI recall score indicates a suggestion for patient recall, and when the processor determines that the AI score is below the threshold, the processor may determine that the AI recall score indicates a suggestion for no patient recall. In some examples, the AI recall score may be a generated, stored, and/or output value or category (an explicit AI recall score). In some examples, the AI recall score may be an implied value or category indicated by the outcome of the comparison (an implicit AI recall score).

At block 810, whether before, during, or after blocks 802-808, the processor may further receive a recall score from a first radiologist (RAD1) via a first workstation (e.g., workstation 602). In an example, the recall score is a binary metric for establishing the classification of the one or more images as BIRAD 0 (additional imaging evaluation needed) or BIRAD 1 or 2 (no patient recall needed). For example, RAD1 may determine that the one or more images are classified as BIRAD 1 (negative) or BIRAD 2 (benign), thus indicating that the patient should not be recalled for further evaluation. Alternatively, RAD1 may determine that the one or more images are classified as BIRAD 0, thus indicating that the patient should be recalled for further evaluation. In either event, the determination to recall or not recall the patient may be received by the first workstation from RAD1. The first workstation may then transmit the determination, as the radiologist recall score, to the processor (e.g., the processor 202 or 212).

At block 812, the processor (e.g., the processor 202 or 212) determines, using the AI recall score and the radiologist (RAD1) recall score, whether to output the one or more images to a second workstation for review by a second radiologist (RAD2). As an example, when the AI recall score and the radiologist recall score conflict, the processor may determine to output the one or more images to the second workstation for review by the second radiologist (RAD2). However, when the AI recall score and the radiologist recall score are the same, the processor may determine not to output the one or more images to the second workstation for review by the second radiologist (RAD2). In some examples, when the AI recall score is an explicit AI recall score, the processor may compare the explicit AI recall score to the radiologist (RAD1) recall score to determine whether the scores match or conflict. In some examples, when the AI recall score is an implicit AI recall score, the processor may determine whether the AI recall score and the radiologist (RAD1) recall score match by, for example, determining whether both the AI score is above a threshold and the radiologist recall score indicates to recall the patient. Similarly, in some examples, when the AI recall score is an implicit AI recall score, the processor may determine whether the AI recall score and the radiologist (RAD1) recall score conflict by, for example, determining whether the AI score is above a threshold and the radiologist recall score indicates not to recall the patient, or determining whether the AI score is below a threshold and the radiologist recall score indicates to recall the patient.

At block 814, the processor (e.g., the processor 202 or 212) generates an indication of the determination at block 812. In some examples, generating the indication includes transmitting the one or more images to the second workstation for review by the second radiologist (e.g., when the AI recall score and the radiologist recall score are the conflict). For example, the processor 202 or 212 may cause the one or more images to be transmitted to the second workstation along with a request (e.g., that may be displayed or otherwise output by the second workstation) for the second radiologist to review the one or more images. The second workstation may, in response, display the one or more images and receive a second radiologist recall score from the second radiologist, which may be a binary metric indicating whether to recall or not recall the patient.

In some examples, generating the indication includes initiating a report generation (e.g., when the AI recall score and the radiologist recall score are the same). For example, when the radiologist recall score indicates the decision to recall the patient, generating the indication may include generating a BIRADS 0 report. In another example, when the AI score is lower than the threshold and the radiologist recall score indicates the decision not to recall the patient, generating the indication may include generating a BIRADS 1 or 2 report. The processor may output the generated report for local display on a workstation or transmit the report to another device (e.g., via the communication network 154) for display or storage on another workstation, computing device, or server.

The processor executing the method of FIG. 8A may execute blocks 812 and 814 using various logic steps based on the various pairings of the AI score and RAD1 recall score. FIGS. 8B-8C illustrate two such examples of steps that may be executed to implement blocks 812 and 814 of FIG. 8A. Further, FIG. 8D illustrates a modified version of the method illustrated by the combination of FIGS. 8A and 8C. Further, FIG. 8E illustrates a modified version of the method illustrated by the combination of FIGS. 8A and 8D.

Turning to FIG. 8B, in block 815, the processor determines whether the radiologist recall score indicates to order a recall. When the radiologist recall score indicates to order a recall, the processor may proceed to block 816 and generate a report (e.g., BIRADS 0 report). When the radiologist recall score indicates not to order a recall (following block 810), the processor may proceed to block 817 and determine whether the AI recall score suggests to recall. When, in block 817, the AI recall score suggests not to recall the patient, the AI recall score and the radiologist recall score match, and the processor may proceed to block 816 to generate a report (e.g., BIRADS 1 or 2 report). When, in block 817, the AI recall score suggests to recall the patient, the AI recall score and the radiologist recall score conflict, and the processor may proceed to block 818 to initiate transmission of the imaging data to a second workstation for review by a second radiologist (as described with respect to block 814 above). In some examples, to initiate transmission of the imaging data, the processor may transmit or cause the images to be transmitted to the second workstation. In some examples, to initiate transmission of the imaging data, the processor transmits an image analysis request to the second radiologist (e.g., to the second workstation or another computing device) that one or more of indicates the request for analysis, identifying the imaging data to be analyzed, and identifies the second radiologist RAD2 (e.g., via a name and/or contact information). In some examples, initiating transmission of the imaging data further includes initiating transmission of AI output from the AI model corresponding to the imaging data. For example, the AI output may include the AI score (e.g., the AI score 554) and/or localization information. The localization information may include, for example, one or more ROIs identified in the imaging data by the model 500. This AI output may assist a second radiologist RAD2 in reviewing the imaging data.

The processor may then receive a second radiologist recall score from the second workstation indicating the assessment of the second radiologist. In block 819, the processor determines whether the second radiologist recall score suggests to recall the patient. When the second radiologist recall score indicates not to order a recall (matching the first radiologist recall score), the processor may proceed to block 816 and generate a report (e.g., BIRADS 1 or 2 report). When the second radiologist recall score indicates to order a recall, the processor may proceed to block 820. In block 820, the processor initiates a consultation request between RAD1 and RAD 2. For example, the processor may transmit a request to one or both radiologists (e.g., via one or both of the first workstation and the second workstations or another computing device) that one or more of indicates the conflict in recall scores, identifying the imaging data, and identifies one or both of the radiologists RAD1 and RAD2 (e.g., via a name and/or contact information).

Turning to FIG. 8C, in block 821, the processor determines whether the radiologist recall score indicates to order a recall. When the radiologist recall score indicates to order a recall, the processor may proceed to block 823 to determine whether the AI recall score also suggests to recall. When, in block 823, the AI recall score also suggests to recall the patient (i.e., the AI recall score and the radiologist recall score match), the processor may proceed to block 816 to generate a report (e.g., BIRADS 0 report). When, in block 823, the AI recall score suggests not to recall the patient (i.e., the AI recall score and the radiologist recall score conflict), the processor may proceed to block 818 to transmit imaging data to a second workstation for review by a second radiologist (as described with respect to block 818 above).

Returning to block 821, when the radiologist recall score indicates not to order a recall, the processor may proceed to block 825 to determine whether the AI recall score also suggests not to recall. When, in block 825, the AI recall score also suggests not to recall the patient (i.e., the AI recall score and the radiologist recall score match), the processor may proceed to block 816 to generate a report (e.g., BIRADS 1 or 2 report). When, in block 825, the AI recall score suggests to recall the patient (i.e., the AI recall score and the radiologist recall score conflict), the processor may proceed to block 818 to initiate transmission of the imaging data to a second workstation for review by a second radiologist (as described with respect to block 818 above). As noted above, in some examples, initiating transmission of the imaging data further includes initiating transmission of AI output from the AI model corresponding to the imaging data.

In block 818, after the image data has been transmitted to the second workstation, the processor may receive a second radiologist recall score from the second workstation indicating the assessment of the second radiologist. In block 827, the processor determines whether the second radiologist recall score suggests to recall the patient. When the second radiologist recall score matches the first radiologist recall score, the processor may proceed to block 816 and generate a report (e.g., BIRADS 0, 1, or 2 report, as indicated by the matching recall scores). When the second radiologist recall score conflicts with the first radiologist recall score, the processor may proceed to block 820. In block 820, the processor initiates a consultation request between RAD1 and RAD 2. For example, the processor may transmit a request to one or both radiologists (e.g., via one or both of the first workstation and the second workstations or another computing device) that one or more of indicates the conflict in recall scores, identifying the imaging data, and identifies one or both of the radiologists RAD1 and RAD2 (e.g., via a name and/or contact information).

Turning to FIG. 8D, the method is generally similar to the method of FIG. 8C, with like steps sharing like label numbers, except that decision block 830 and block 810 are included in the method. In particular, in the method of FIG. 8D, before proceeding to block 810, the processor determines whether the AI score (e.g., the score 554 output by the model 500) is above a lower threshold. When the AI score is above the lower threshold, the processor transmits a request for an assessment by the first radiologist (e.g., at the first workstation). In response to this request, in block 810, the first radiologist recall score may be received (as described in further detail above). When the AI score is below the lower threshold, the processor may proceed to block 816 to generate a report (e.g., BIRADS 1 or 2 report). Accordingly, those sets of images that the AI model determines has a very low likelihood of malignancy can be filtered out and not sent for radiologist analysis, improving efficiencies and freeing up resources for other analyses.

Turning to FIG. 8E, a method 840 is illustrated. In some examples, the method 840 may be inserted into the method of FIG. 8D (e.g., before block 830), and may start at block 842. Steps in the method 840 sharing like label numbers with steps in FIGS. 8A-8D may be similar. For example, a processor (e.g., the processor 202 or 212) may execute blocks 802 to 808 (or 802 to 806) of FIG. 8A (as described above), and then proceed to block 842 in FIG. 8E. In block 842, the processor determines whether the AI score (e.g., the score 554 output by the model 500) is above an upper threshold. In some examples, when the AI score is below the upper threshold, the processor may proceed to block 830 of FIG. 8D. In other examples, when the AI score is below the upper threshold, the method may end.

When the AI score is above the upper threshold, at block 844, the processor initiates transmission of imaging data to both a first radiologist (e.g., at the first workstation) and a second radiologist (e.g., at a second workstation). In some examples, to initiate transmission of the imaging data, the processor may transmit or cause the images to be transmitted to the first and second workstation. In some examples, to initiate transmission of the imaging data, the processor transmits an image analysis request to the first and second radiologist (e.g., to workstations or devices thereof) that one or more of indicates the request for analysis, identifying the imaging data to be analyzed, and identifies the first radiologist RAD1 and/or the second radiologist RAD2 (e.g., via a name and/or contact information). As noted above, in some examples, initiating transmission of the imaging data further includes initiating transmission of AI output from the AI model corresponding to the imaging data. Accordingly, in some examples, the first and second workstation also receive the AI output corresponding to the imaging data.

In response to this transmission, at block 846, the processor receives the first radiologist recall score and the second radiologist recall score (e.g., in a similar manner as the processor may receive other radiologist scores) may be received (as described in further detail above).

In block 848, the processor determines whether the second radiologist recall score matches the first radiologist recall score. When the second radiologist recall score matches the first radiologist recall score, the processor may proceed to block 816 and generate a report (e.g., BIRADS 0, 1, or 2 report, as indicated by the matching recall scores). When the second radiologist recall score conflicts with the first radiologist recall score, the processor may proceed to block 820. In block 820, the processor initiates a consultation request between RAD1 and RAD 2.

Accordingly, the method 840 enables initiation of independent review by two radiologists when the AI score indicates a high likelihood of a malignancy in the imaging data (e.g., based on the AI score being above the upper threshold).

In some examples, the method 840 (e.g., starting with block 842) may be inserted downstream of block 830 in FIG. 8D (e.g., along the “Y” branch leading to block 810). In such examples, the processor may proceed to block 810 of FIG. 8D when the processor determines in block 842 of FIG. 8E that the AI score is not above the upper threshold. In some examples, block 830 and 842 may be performed in parallel or partially in parallel.

Accordingly, with reference to FIGS. 8A, 8D, and 8E, a method is provided that enables: (a) initiating independent review by two radiologists when the AI score is above an upper threshold, (b) initiating a report (e.g., BIRADS 1 or 2) without initiating radiologist review when the AI score is below a lower threshold, and (c) determining whether to initiate an independent review by a second radiologist based on whether an AI recall score and radiologist recall score conflict. In some examples, this method may be repeated for imaging data of a plurality of patients. Accordingly, first imaging data for a first patient may have a first AI score above the upper threshold and the method may include initiating independent review by two radiologists; second imaging data for a second patient may have a second AI score below the lower threshold and the method may include generating a report without initiating radiologist review; and third imaging data for a third patient may have a third AI score between the upper and lower thresholds, and the method may include initiating a first radiologist's review, determining whether an AI recall score for the third imaging data conflicts with the first radiologist's recall score of the third imaging data, and initiating a further review by a second radiologist when such a conflict is determined.

In some examples, feature (a) is performed independently of (b) and/or (c). For example, the processor may perform feature (a) without also evaluating or taking action based on whether the AI score is below a lower threshold, and/or the processor may perform feature (a) without also evaluating or taking action based on whether the AI recall score and the radiologist recall score conflict. Similarly, in some examples, feature (b) is performed independently of (a) and/or (c). For example, the processor may perform feature (b) without also evaluating or taking action based on whether the AI score is above an upper threshold, and/or the processor may perform feature (b) without also evaluating or taking action based on whether the AI recall score and the radiologist recall score conflict.

Although the above systems and processes are generally described with respect to analysis of breast tissue-related images, in some examples, the systems and processes are applied to images of other types of tissue (e.g., lung tissue, prostate tissue, etc.). In some such examples, the reports generated may take a different form than a BIRADS report, such as a form specific to the particular type of tissue or generic to various types of tissue. Additionally, in some such examples, the actions taken as a result of the determinations may vary. For example, in addition to or instead of one or more of the steps that indicate to generate a report that indicates to initiate patient recall, the report may indicate to take another action particular for the type of tissue under analysis.

Additionally, the systems and methods described herein may implement AI model-based quality review for clinician assessments, which include assessments by, for example, an imaging technologist (who operates imaging machines to capture medical images), a physician (e.g., radiologist or pathologist), or the like. For example, the system 104 (e.g., the processor 202 or 212) may transmit imaging data including one or more medical images of a patient tissue (e.g., of soft tissue (e.g., muscles, fat, blood vessels, nerves, tendons, organs, etc.) or hard tissue (e.g., bone, teeth, etc.)) to an AI model (e.g., as described with respect to block 804). Such medical images may be captured for the purpose of assessing or diagnosing various ailments or abnormalities, such as, for example, cancer, bone fractures, hemorrhages, etc. The system 104 may receive an AI output from the AI model, where the AI output indicates an assessment of the imaging data by the AI model. For example, the AI output may be the AI score described with respect to block 806 or the AC recall score described with respect to block 808. In some examples, the AI score indicates a likelihood or determination of an ailment or abnormality (e.g., presence of cancer, bone fracture, hemorrhage, etc.). In some examples, the AI score indicates a quality of the image in terms of image characteristics (e.g., brightness, blur, etc.) and/or positioning of a patient with respect to the imaging device and the medical image(s) captured (e.g., indicating whether the imaging data is sufficient for review by a physician to perform an assessment). The system 104 may further compare the AI output received from the AI model with a clinician assessment of the imaging data by a clinician. The clinician assessment may be an indication of a likelihood of an abnormality in the patient tissue (e.g., a malignancy, bone fracture, hemorrhage, etc.) or may be the recall score as described with respect to block 810. The clinician assessment may be of the same type of assessment as the AI score. For example, when the AI score indicates a quality of the image, the clinician assessment may also be an indication of the quality. In some examples, the clinician assessment is implied by an action. For example, when an imaging technologist accepts one or more captured medical image(s) (e.g., saves to the system and/or transmits them to another clinician for assessment), such acceptance may be interpreted by the system 104 as a clinician assessment that indicates the one or more medical images are of a certain sufficient quality level for use in assessment by a physician. The system 104 may further generate an indication of a quality of the clinician assessment of the imaging data based on the comparing of the AI output with the clinician assessment.

The indication may be or include the indication of the determination described with respect to the block 814, the initiation of a consultation as described with respect to block 820, the transmission of the one or more images to a second radiologist workstation as described with respect to block 818, and/or the generation of a report as described with respect to block 816. In some examples, when the indication is or includes the generation of a report, the indication of the quality may indicate that the clinician assessment is of a first (high) quality, and when the indication is or includes the initiation of a consultation or transmission of the one or more images to a second workstation (or a determination to do so), the indication of the quality may indicate that the clinician assessment is of a second (low) quality. In some examples, when the comparison indicates that the AI output matches or is similar to the clinician assessment, the indication of the quality may indicate that the clinician assessment is of a first (high) quality, and when the comparison indicates that the AI output does not match or is dissimilar to the clinician assessment, the indication of the quality may indicate that the clinician assessment is of a second (low) quality.

The following example describes a study of the potential effectiveness of some examples of the methods of the combination of FIGS. 8A and 8C described above for evaluating breast tissue imagery using an AI model, as measured by additional cancers detected. In this example, the method for evaluating breast tissue imagery using an AI model (e.g., the system 104 or the model 708) was implemented across a single group practice covering eight clinical sites. Performance metrics including the number of cancers detected, cancer detection rates (CDR), abnormal interpretation rates (AIR), and positive predictive value (PPV1) from 40,532 screening mammograms interpreted by six radiologists were collected without and with the implementation of the example method for evaluating breast tissue imagery using an AI model. The six radiologists had a wide range of experience (from <5 years to >25 years in practice), and each radiologist read at least 500 screening mammograms during the study.

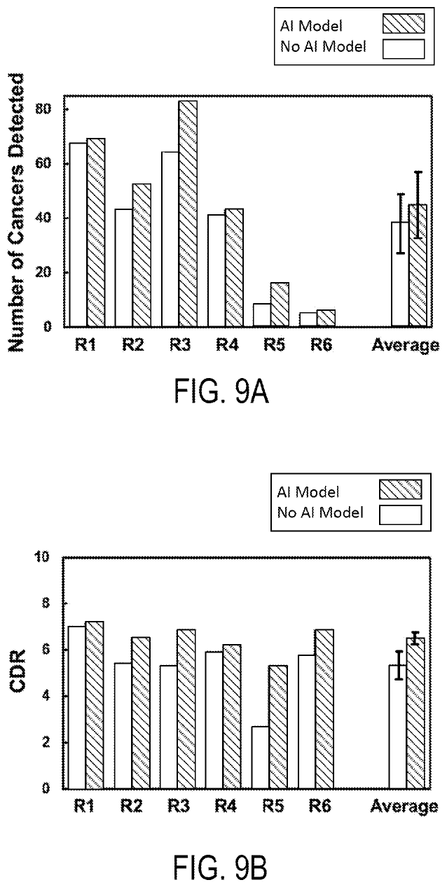

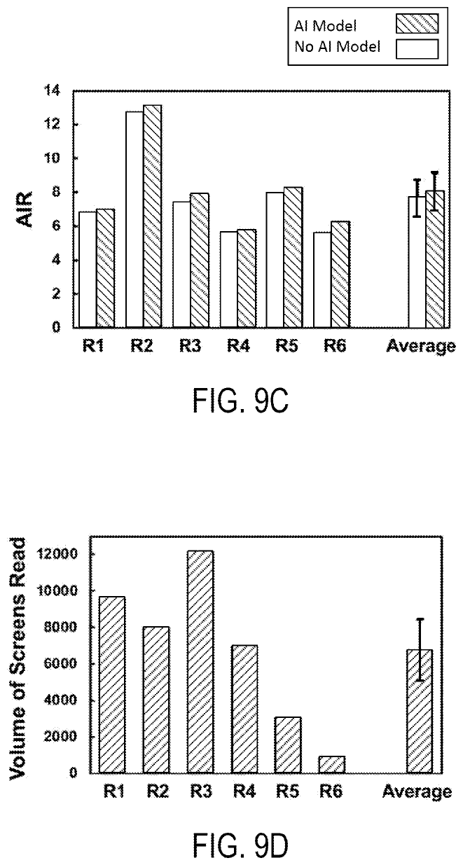

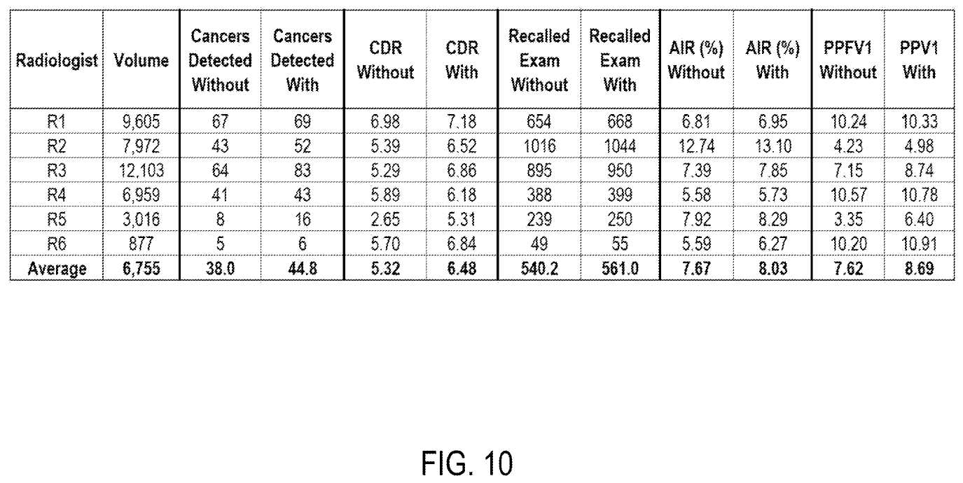

Use of the example method for evaluating breast tissue imagery using an AI model breast tissue imagery evaluation method led to detection of an additional 41 cancers (FIG. 9A) that would have otherwise been missed, in addition to the 228 cancer that were found via initial interpretations. Across 6 radiologists, the CDR increased from 5.3 to 6.5 (p<0.05) with the AI model method (FIG. 9B). The radiologist also demonstrated increased AIR from 7.7% to 8.0% (p<0.05) (FIG. 9C). However, the PPV1 of the additional recalled exams was 32.8%, which is significantly higher than the PPV1 without AI model-driven breast tissue imagery evaluation method (7.0%), suggesting that there is a greater fraction of cancers within the additional recalled claims. A total of 40,532 screening mammograms were interpreted by the six radiologists during the study (FIG. 9D). These results are presented in the table in FIG. 10. That is, the chart in FIG. 10 shows performance metrics by the six radiologists collected without and with the implementation of the noted method for evaluating breast tissue imagery using an AI model.

AI model-driven selection of exams for concurrent evaluation increased the number of cancers detected for every radiologist, with a small increase in AIR in a real-world high-volume community practice.

### EXAMPLES

Example 1: A method, apparatus, and/or non-transitory computer-readable medium storing processor-executable instructions for evaluating patient tissue imagery using an artificial intelligence (AI) model, comprising: receiving one or more images of tissue of a patient; transmitting the one or more images of the tissue to the AI model; generating an AI score output from the AI model, wherein the AI score assesses the one or more images of the tissue for a likelihood of one or more malignancies; comparing, by a processor, the AI score to a threshold to determine an AI recall score; receiving, by the processor from a first physician via a first workstation, a physician recall score for the one or more images indicating an assessment by the first physician; determining, by the processor based on the AI recall score and the physician recall score, whether to output the one or more images to a second workstation for review by a second physician; and generating, by the processor, an indication of the determination.
Example 2: The method, apparatus, and/or non-transitory computer readable medium of Example 1, wherein the physician recall score is a binary score indicating a decision to recall the patient or not to recall the patient.
Example 3: The method, apparatus, and/or non-transitory computer readable medium of any of Examples 1 or 2, further comprising: determining whether the AI score is above a lower threshold; and in response to the AI score being above the lower threshold, transmitting a request for the assessment by the first physician, wherein the physician recall score is received in response to the request.
Example 4: The method, apparatus, and/or non-transitory computer readable medium of any of Examples 1 to 3, further comprising: determining whether the AI score is below an upper threshold; and in response to the AI score being below the upper threshold, transmitting the request for the assessment by the first physician, wherein the physician recall score is received in response to the request.
Example 5: The method, apparatus, and/or non-transitory computer readable medium of any of Examples 1 to 4, wherein the indication of the determination includes: initiating a report generation when the physician recall score indicates the decision to recall the patient; or initiating a report generation when the AI score is lower than the threshold and the physician recall score indicates the decision not to recall the patient.
Example 6: The method, apparatus, and/or non-transitory computer readable medium of any of Examples 1 to 4, wherein the indication of the determination includes: initiating transmission of the one or more images to the second workstation for review by the second physician when the AI score is higher than the threshold and the physician recall score indicates the decision not to recall the patient.
Example 7: The method, apparatus, and/or non-transitory computer readable medium of any of Examples 1 to 4, wherein the indication of the determination includes: initiating a report generation when the AI score is lower than the threshold and the physician recall score indicates the decision not to recall the patient; or initiating a report generation when the AI score is higher than the threshold and the physician recall score indicates the decision to recall the patient.
Example 8: The method, apparatus, and/or non-transitory computer readable medium of any of Examples 1 to 4, wherein the indication of the determination includes: initiating transmission of the one or more images to the second workstation for review by the second physician when the AI score is higher than the threshold and the physician recall score indicates the decision not to recall the patient; or initiating transmission of the one or more images to the second workstation for review by the second physician when the AI score is lower than the threshold and the physician recall score indicates the decision to recall the patient.
Example 9: The method, apparatus, and/or non-transitory computer readable medium of any of Examples 1 to 4, 6, or 8, further comprising: receiving, from the second physician via the second workstation, a second physician recall score for the one or more images indicating an assessment by the second physician; and initiating a report generation when the second physician recall score is the same as the physician recall score received from the first physician.
Example 10: The method, apparatus, and/or non-transitory computer readable medium of any of Examples 1 to 4, 6, or 8, further comprising: receiving, from the second physician via the second workstation, a second physician recall score for the one or more images indicating an assessment by the second physician; and transmitting a consultation request when the second physician recall score is different than the physician recall score received from the first physician.
Example 11: The method, apparatus, and/or non-transitory computer readable medium of any of Examples 6 or 8, wherein initiating transmission of the one or more images to the second workstation for review by the second physician further includes initiating transmission of AI output generated by the AI model corresponding to the one or more images, the AI output including the AI score, localization information, or both the AI score and the localization information.
Example 12: The method, apparatus, and/or non-transitory computer readable medium of any of Examples 1 to 4, 6, or 8, 1-11, wherein the patient tissue images are breast tissue images, lung tissue images, or prostate tissue images.
Example 13: A method, apparatus, and/or non-transitory computer-readable medium storing processor-executable instructions for evaluating patient tissue imagery using an artificial intelligence (AI) model, comprising: receiving imaging data of tissue of a plurality of patients; transmitting the imaging data for each patient to the AI model; generating a respective AI score output from the AI model for the imaging data for each patient, wherein each AI score assesses the imaging data of the tissue for a likelihood of one or more malignancies; comparing, by a processor, the AI scores to an upper threshold; and in response to a first AI score of the AI scores being above the upper threshold: transmitting, by the processor to a first workstation, a request for an assessment by a first physician of imaging data corresponding to the first AI score, and transmitting, by the processor to a second workstation, a request for an assessment by a second physician of the imaging data corresponding to the first AI score.
Example 14: The method, apparatus, and/or non-transitory computer readable medium of Example 13, further comprising: comparing, by a processor, the AI scores to a lower threshold; and in response to a second AI score of the AI scores being below the lower threshold, initiating a report generation for the imaging data corresponding to the second AI score and bypassing physician review.
Example 15: The method, apparatus, and/or non-transitory computer readable medium of any of Examples 13 to 14, further comprising: in response to a third AI score of the AI scores being below the upper threshold and above the lower threshold, transmitting, by the processor to the first workstation, a request for an assessment by the third physician of the imaging data corresponding to the third AI score.
Example 16: The method, apparatus, and/or non-transitory computer readable medium of Example 15, further comprising: comparing the third AI score to a threshold to determine an AI recall score; receiving, from the first physician via the first workstation, a physician recall score indicating an assessment by the first physician of the imaging data corresponding to the third AI score; determining, based on the AI recall score and the physician recall score, whether to output the imaging data corresponding to the third AI score to a second workstation for review by a second physician; and generating an indication of the determination.
Example 17: The method, apparatus, and/or non-transitory computer readable medium of Example 16, wherein the indication of the determination includes: initiating a report generation when the physician recall score indicates the decision to recall the patient; or initiating a report generation when the third AI score is lower than the threshold and the physician recall score indicates the decision not to recall the patient.
Example 18: The method, apparatus, and/or non-transitory computer readable medium of Example 16, wherein the indication of the determination includes: initiating transmission of the imaging data corresponding to the third AI score to the second workstation for review by the second physician when the third AI score is higher than the threshold and the physician recall score indicates the decision not to recall the patient.
Example 19: The method, apparatus, and/or non-transitory computer readable medium of Example 16, wherein the indication of the determination includes: initiating a report generation when the third AI score is lower than the threshold and the physician recall score indicates the decision not to recall the patient; or initiating a report generation when the third AI score is higher than the threshold and the physician recall score indicates the decision to recall the patient.
Example 20: The method, apparatus, and/or non-transitory computer readable medium of Example 16, wherein the indication of the determination includes: initiating transmission of the imaging data corresponding to the third AI score to the second workstation for review by the second physician when the third AI score is higher than the threshold and the physician recall score indicates the decision not to recall the patient; or initiating transmission of the imaging data corresponding to the third AI score to the second workstation for review by the second physician when the third AI score is lower than the threshold and the physician recall score indicates the decision to recall the patient.
Example 21: The method, apparatus, and/or non-transitory computer readable medium of any of Examples 13 to 20, wherein the imaging data comprises breast tissue images, lung tissue images, or prostate tissue images.
Example 22: A method, apparatus, and/or non-transitory computer-readable medium storing processor-executable instructions for evaluating patient tissue imagery using an artificial intelligence (AI) model, comprising: transmitting one or more images of patient tissue to an AI model; receiving an AI output from the AI model, where the AI output indicates an assessment of the imaging data by the AI model; comparing the AI output received from the AI model with a physician assessment of the imaging data by a physician; and generating an indication of a quality of the physician assessment of the imaging data based on the comparing of the AI output with the physician assessment.
Example 23: The method, apparatus, and/or non-transitory computer readable medium of Example 22, wherein the AI output is indicative of a likelihood of an abnormality in the patient tissue.
Example 24: The method, apparatus, and/or non-transitory computer readable medium of Example 22 or 23, wherein the AI output is an AI recall score, and the physician assessment is a physician recall score.
Example 25: The method, apparatus, and/or non-transitory computer readable medium of any of Examples 22 to 24, wherein the indication of the quality includes an indication of whether to output the one or more images to a workstation for review by a second physician.

The present disclosure includes the description of one or more embodiments, and it should be appreciated that many equivalents, alternatives, variations, and modifications, aside from those expressly stated, are possible and within the scope of the disclosure and claims.

1. A method for evaluating patient tissue imagery using an artificial intelligence (AI) model, the method comprising:
receiving one or more images of tissue of a patient;
transmitting the one or more images of the tissue to the AI model;
generating an AI score output from the AI model, wherein the AI score assesses the one or more images of the tissue for a likelihood of one or more malignancies;
comparing, by a processor, the AI score to a threshold to determine an AI recall score;
receiving, by the processor from a first physician via a first workstation, a physician recall score for the one or more images indicating an assessment by the first physician;
determining, by the processor based on the AI recall score and the physician recall score, whether to output the one or more images to a second workstation for review by a second physician; and
generating, by the processor, an indication of the determination.

2. The method of claim 1, wherein the physician recall score is a binary score indicating a decision to recall the patient or not to recall the patient.

3. The method of claim 1, further comprising:
determining whether the AI score is above a lower threshold; and
in response to the AI score being above the lower threshold, transmitting a request for the assessment by the first physician, wherein the physician recall score is received in response to the request.

4. The method of claim 1, further comprising:
determining whether the AI score is below an upper threshold; and
in response to the AI score being below the upper threshold, transmitting a request for the assessment by the first physician, wherein the physician recall score is received in response to the request.

5. The method of claim 1, wherein the indication of the determination includes:
initiating a report generation when the physician recall score indicates a decision to recall the patient; or
initiating a report generation when the AI score is lower than the threshold and the physician recall score indicates a decision not to recall the patient.

6. The method of claim 1, wherein the indication of the determination includes:
initiating transmission of the one or more images to the second workstation for review by the second physician when the AI score is higher than the threshold and the physician recall score indicates a decision not to recall the patient.

7. The method of claim 1, wherein the indication of the determination includes:
initiating a report generation when the AI score is lower than the threshold and the physician recall score indicates a decision not to recall the patient; or
initiating a report generation when the AI score is higher than the threshold and the physician recall score indicates a decision to recall the patient.

8. The method of claim 1, wherein the indication of the determination includes:
initiating transmission of the one or more images to the second workstation for review by the second physician when the AI score is higher than the threshold and the physician recall score indicates a decision not to recall the patient; or
initiating transmission of the one or more images to the second workstation for review by the second physician when the AI score is lower than the threshold and the physician recall score indicates a decision to recall the patient.

9. The method of claim 1, further comprising:
receiving, from the second physician via the second workstation, a second physician recall score for the one or more images indicating an assessment by the second physician; and
initiating a report generation when the second physician recall score is the same as the physician recall score received from the first physician.

10. The method of claim 1, further comprising:
receiving, from the second physician via the second workstation, a second physician recall score for the one or more images indicating an assessment by the second physician; and
transmitting a consultation request when the second physician recall score is different than the physician recall score received from the first physician.

11. The method of claim 8, wherein initiating transmission of the one or more images to the second workstation for review by the second physician further includes initiating transmission of AI output generated by the AI model corresponding to the one or more images, the AI output including the AI score, localization information, or both the AI score and the localization information.

12. The method of claim 1, wherein the patient tissue images are breast tissue images, lung tissue images, or prostate tissue images.

13. A system for evaluating patient tissue imagery using an artificial intelligence (AI) model, the system comprising:
a memory;
a communication interface;
a processor coupled to the memory and to the communication interface, the processor configured to:
receive one or more images of tissue of a patient;
transmit the one or more images of the tissue to the AI model;
generate an AI score output from the AI model, wherein the AI score assesses the one or more images of the tissue for a likelihood of one or more malignancies;
compare the AI score to a threshold to determine an AI recall score;
receive, via the communication interface, from a first physician via a first workstation, a physician recall score for the one or more images indicating an assessment by the first physician;
determine, based on the AI recall score and the physician recall score, whether to output the one or more images to a second workstation for review by a second physician; and
generate an indication of the determination.

14. (canceled)

15. The system of claim 13, the processor further configured to:
determining whether the AI score is above a lower threshold or below an upper threshold; and
in response to the AI score being above the lower threshold or below the upper threshold, transmitting a request for the assessment by the first physician, wherein the physician recall score is received in response to the request.

16.-17. (canceled)

18. The system of claim 13, wherein, to generate the indication of the determination, the processor is further configured to:
initiate transmission of the one or more images to the second workstation for review by the second physician when the AI score is higher than the threshold and the physician recall score indicates a decision not to recall the patient.

19. The system of claim 13, wherein, to generate the indication of the determination, the processor is further configured to:
initiate a report generation when the AI score is lower than the threshold and the physician recall score indicates a decision not to recall the patient; or
initiate a report generation when the AI score is higher than the threshold and the physician recall score indicates a decision to recall the patient.

20. The system of claim 13, wherein, to generate the indication of the determination, the processor is further configured to:
initiate transmission of the one or more images to the second workstation for review by the second physician when the AI score is higher than the threshold and the physician recall score indicates a decision not to recall the patient; or
initiate transmission of the one or more images to the second workstation for review by the second physician when the AI score is lower than the threshold and the physician recall score indicates a decision to recall the patient.

21.-24. (canceled)

25. A nontransitory computer readable medium comprising instructions that, when executed by a processor, cause the processor to:
receive one or more images of tissue of a patient;
transmit the one or more images of the tissue to an artificial intelligence (AI) model;
generate an AI score output from the AI model, wherein the AI score assesses the one or more images of the tissue for a likelihood of one or more malignancies;
compare the AI score to a threshold to determine an AI recall score;
receive, from a first physician via a first workstation, a physician recall score for the one or more images indicating an assessment by the first physician;
determine, based on the AI recall score and the physician recall score, whether to output the one or more images to a second workstation for review by a second physician; and
generate an indication of the determination.

26. (canceled)

27. The nontransitory computer readable medium of claim 25, the instructions further configured to cause the processor to:
determining whether the AI score is above a lower threshold or below an upper threshold; and
in response to the AI score being above the lower threshold or below the upper threshold, transmitting a request for the assessment by the first physician, wherein the physician recall score is received in response to the request.

28.-31. (canceled)

32. The nontransitory computer readable medium of claim 25, wherein, to generate the indication of the determination, the instructions are further configured to cause the processor to:
initiate transmission of the one or more images to the second workstation for review by the second physician when the AI score is higher than the threshold and the physician recall score indicates a decision not to recall the patient; or
initiate transmission of the one or more images to the second workstation for review by the second physician when the AI score is lower than the threshold and the physician recall score indicates a decision to recall the patient.

33.-67. (canceled)
